(19) United States
(12) Patent Application Publication
(10) Pub. No.: US 2005/0107451 A1
(43) Pub. Date: May 19, 2005

(54) Methods for treating conditions, disorders, or diseases involving cell death

(76) Inventor: Stuart D. Portbury, Durham, NC (US)

Correspondence Address: HUTCHISON & MASON PLLC, PO box 31686, RALEIGH, NC, 27612, US

(21) Appl. No.: 10/495,323

(22) PCT Filed: Dec. 20, 2002
(86) PCT No.: PCT/US02/40643

Related U.S. Application Data
(60) Provisional application No. 60/342,712, filed on Dec. 20, 2001.

(51) Int. Cl. A61K 31/41

(52) U.S. Cl. 514/381

(57) ABSTRACT

The present invention relates to methods for the treatment of conditions, disorders, or diseases involving cell death. The invention also relates to methods of preventing, inhibiting, or treating cells predisposed to undergo cell death or in the process of undergoing cell death. The invention further relates to methods of preventing, inhibiting, or treating cell death.

### FIELD OF THE INVENTION

The present invention relates to methods for the treatment of conditions, disorders, or diseases involving cell death. The invention relates to methods of preventing, inhibiting, or treating cells predisposed to undergo cell death or in the process of undergoing cell death by administering an effective amount of a protective compound. An effective amount of a protective compound prevents, delays, or rescues the cell from death relative to a corresponding cell into which a compound has not been administered.

### BACKGROUND OF THE INVENTION

It is widely recognized that at least two distinct cell death mechanisms exist for mammalian cells. These two mechanisms are necrosis and apoptosis, and are significant components of numerous conditions, disorders and disease states.

Necrosis plays an important physiologic role in signaling the presence of certain conditions. When cells die as a result of necrosis, the dying cells release substances that activate the body's immune response in a local, and in some cases widespread, reaction to the necrosis-inducing condition. This response is important in, for example, bacterial infection.

Experimental evidence in a wide variety of cells throughout the body has revealed that every cell can initiate a program of self-destruction, called apoptosis. This program can be initiated by a wide variety of natural and unnatural events. There are at least four distinct pathways for executing this program of cell death, and it is virtually certain that dozens, if not hundreds, of different intracellular biochemical cascades interact with each pathway. It is equally likely that certain cell types, such as cells in the heart or neurons, will utilize specialized signaling pathways that are not generally represented elsewhere in the body. However, since cell death is neither always necessary nor desired, it would be desirable to manipulate the manner in which cells start their death process. In some circumstances, preventing, delaying, or rescuing cells from death would either alleviate the disease or allow more time for definitive treatment to be administered to the patient. An example of this situation is brain cell death caused by ischemic stroke: preventing, delaying, or rescuing cells from death until the blood supply to the brain could be restored would greatly reduce, if not eliminate, the possibility of a person's death and/or long-term disability from stroke (Lee J M, et al. Nature 1999, 399(supp): A7-A14; Tarkowski E, et al. Stroke 1999, 30(2): 321-7; Pulera M R, et al. Stroke 1998, 29(12): 2622-30). In still other circumstances, the failure of cells to die may itself lead to disease such as cancer (Hetts S W. JAMA 1998, 297(4): 300-7).

Cell death plays an important role in the normal function of mammalian organisms. While it may seem counterintuitive for cells to have death as a normal part of their life cycle, controlled and physiologically appropriate cell death is important in regulating both the absolute and relative numbers of cells of a specific type. (Hetts S W. JAMA 1998, 297(4): 300-7; Garcia I, et al. Science 1992, 258(5080): 302-4). When the mechanism of apoptosis does not function properly and normal cell death does not occur, the resulting disease is characterized by unregulated cellular proliferation, as occurs in a neoplastic disease or an autoimmune disease (Hetts S W. JAMA 1998, 297(4): 300-7; Yachida M, et al. Clin Exp Immunol 1999, 116(1): 140-5).

One method for regulating cell death involves manipulating the threshold at which the process of cell death begins. This threshold varies significantly by cell type, tissue type, the type of injury or insult suffered by the cell, cellular maturity, and the physiologic conditions in the cell's environment (Steller H., Science 1995, 267(5203): 1445-9). Although it is probable that certain cellular injuries or insults irrevocably induce death, lesser injuries or insults may begin the dying process without inducing irreversible cell death. What constitutes a lesser injury or insult may vary tremendously with changes in the factors influencing that cell's death threshold. The ability to alter a cell's threshold for responding to an injury or insult, that is, to either promote or discourage cell death, would be a desirable goal for the treatment of conditions involving cell death. The ability to better control cell death, by either discouraging or promoting the mechanisms of cell death, would be an important invention for ameliorating disease (U.S. Pat. Nos. 5,925,640; 5,786,173; 5,858,715; 5,856,171).

Recent evidence suggests that the mechanisms of cellular death may be more complex than the two discrete pathways of apoptosis and necrosis. Examples of this evidence may be found in the central nervous system (CNS). In the complex CNS cellular environment, both necrosis and apoptosis are observed with commonly studied conditions, disorders, or diseases such as focal ischemia, global ischemia, toxic insults, prolonged seizures, excitotoxicity, and traumatic brain injury. In some reports, both apoptosis and necrosis have been described (Choi W S, et al. J Neurosci Res 1999, 57(1): 86-94; Li Y, et al. J Neurol Sci 1998, 156(2): 119-32; Lee J-M, et al. Nature 1999, 399(supp): A8-A14; Baumgartner W A, et al. Ann Thorac Surg 1999, 67(6): 1871-3; Fujikawa D G, et al. Eur J Neurosci 1999, 11(5): 1605-14; Gwag B J, et al Neuroscience 1999, 90(4): 1339-48; Mitchell I J, et al. 1998, 84(2): 489-501; Nakashima K, et al. J Neurotrauma 1999, 16(2): 143-51; Ginsburg, Md. Cerebrovascular Disease: Pathophysiology, Diagnosis, and Management 1998 Ch 42; Rink A D, et al. Soc Neurosci Abstr 1994, 20:250(Abstract)). Similar observations also occurred with brain tumor cells. (Maurer B J, et al. J Natl Cancer Inst 1999, 91(13): 1138-46). Other investigators found that neurons die by either apoptosis or necrosis under different environmental conditions (Taylor D L, et al. Brain Pathol 1999, 9(1): 93-117). There also are reports of a unique type of neuronal cell death following stroke. This new type of cell death has features common to both necrosis and apoptosis (Fukuda T, et al. Neurosci Res 1999, 33(1): 49-55). Other investigators believe that neuronal cell death is best represented by a continuum between apoptosis and necrosis, possibly mediated by calcium levels (Lee J-M, et al. 1999, 399(supp): A7-A14), or a combination of direct ischemic damage followed by indirect damage from excitotoxicity and loss of inteneuronal connections (Martin L J, et al. Brian Res Bull 1998, 46(4): 281-309). Further complicating the picture of neuronal cell death is the observation that the death of one or more neurons in one region of the brain can induce the death of neurons in other brain regions. This phenomenon has been observed with stroke as described above (Martin L J, et al. Brain Res Bull 1998, 46(4): 281-309) as well as neuronal cell death induced by the withdrawal of growth factors (Ryu B R, et al. J Neurobiol 1999, 39(4): 536-46). Given the complex nature of actions and interactions among the many physiologic and molecular forces in brain tissue, and the different abilities of many substances acting either alone or in combination to induce cellular injury or death, it is difficult to determine with any degree of certainty if a nerve cell death process is due to apoptosis or necrosis (Graham D I, Greenfield's Neuropathology Ch 3 1997).

Despite the challenges in classifying the mechanism of cellular death, there is broad agreement that most, if not all, cells share common features in their death mechanisms (see, e.g., Lee J. M., et al., Nature 1999, 399 (supp): A7-A14).

Several factors have been reported to inhibit the cell death pathway. One of the best-known factors is the gene product bcl-2 (Adams J M, et al. Science 1998, 281(5381): 1322-6; Vaux D L, et al. Proc Natl Acad Sci 1993, 90(3): 786-9; U.S. Pat. No. 5,856,171 and references cited therein). Expression of bcl-2 is believed to regulate apoptotic death in neurons, kidney, heart, liver, blood and skin cells under experimental conditions. In addition to regulating death by apoptosis, bcl-2 is believed to regulate death caused by non-apoptotic mechanisms. Factors related to bcl-2 have been shown to be over-expressed in cancer and autoimmune conditions, disorders, or diseases (U.S. Pat. No. 5,856,171 and references cited therein). Other related factors acting on the same pathway as bcl-2 also delay or prevent cell death.

In the brain, several factors have been shown to influence the cell death pathway. In excitotoxic injury to neurons, it was shown that lithium or bcl-2 each individually protected neurons against cell death (Nonaka S, et al. Proc Natl Acad Sci 1998, 95(5): 2642-7; Behl C, et al. Biochem Biophys Res Commun 1993, 197(2): 949-56). During ischemic injury to neurons, it was shown that nerve growth factor (NGF) and bcl-2 individually offered protection against neuronal death (Guegan C, et al. Neurobiol Dis 1999, 6(3): 180-9; Linnik M D, et al. Stroke 1995, 26(9): 1670-4).

Factors acting to prevent cell death do not act solely in the brain. In the heart, increased tolerance to non-lethal ischemic injury was associated with an increased expression of the bcl-2 gene, suggesting that bcl-2 was involved in protecting the cardiac muscle cells against ischemic injury (Maulik N, et al. Ann NY Acad Sci 1999, 874:401-11). This same study demonstrated that lower levels of bcl-2 expression were associated with higher rates of cardiac cell death. A similar result was found for mechanical injury to heart papillary muscle cells.

Recently, it has been demonstrated that bcl-2 prevented cell death in a brain ischemia model (Guegan C, et al. Neurobiol Dis 1999, 6(3): 180-9; Linnik M D, et al. Stroke 1995, 26(9): 1670-4). It was shown that the activity of bcl-2 to prevent neuronal death was consistently demonstrated across several different physiologic insults. It also has been demonstrated that the distinction between apoptotic death and necrotic death is open to question, so the possibility exists that bcl-2 can prevent or delay the necrotic cell death pathway, the apoptotic cell death pathway or perhaps an as yet undemonstrated cell death pathway.

Preventing cell death is an important medical goal. Several types of mammalian cells, most notably neurons and cardiac muscle cells, have limited if any capacity to regenerate. Preventing the death of these cells from conditions such as heart attack, stroke, shock, infection, cancer, Alzheimer's disease or traumatic injury, to name a few, would be an important medical advance as the heart and brain cannot grow sufficient cells to replace those cells lost to disease or infection.

In addition to preventing cell death, delaying and/or rescuing cells from cell death is also an important medical goal. In many pathological conditions where there is an expectation that the disease will be successfully treated, such as many types of infection, hypoxia, ischemia or metabolic disturbances, delaying cell death would allow the pathological condition to be treated without permanent damage to the cells. In other words, the cells may be put into a suspended state from which they could successfully be rescued and emerge with their normal function intact.

### SUMMARY OF THE INVENTION

The present invention relates to the discovery of three compounds that exhibit protective activity against cell death, in particular neuroprotective activity. These three compounds are as follows: 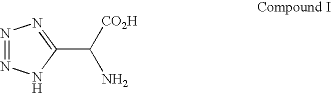
Compound I of the present invention, which exhibits protective activity, is (RS)-(Tetrazol-5-yl)glycine. 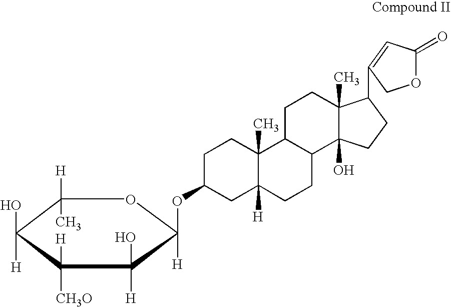
Compound II of the present invention, which exhibits protective activity, is neriifolin or (3β,5β)-3-[(6-Deoxy-3-O-methyl-α-L-glucopyranosyl)oxyl]-14-hydroxycard-20(22)-enolide. 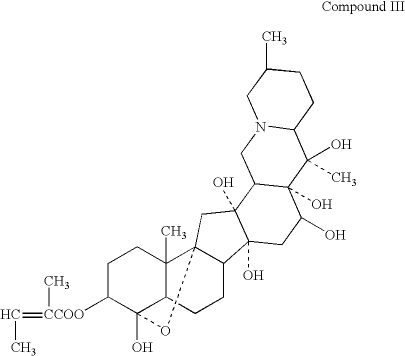
Compound III of the present invention, which exhibits protective activity, is cevadine or (Z)-4α,9-Epoxycevane-3β,4,12,14,16β,17,20-heptol 3-(2-methyl-2-butenoate)

By exhibiting protective activity against cell death, these three compounds when contacted with a cell either predisposed to undergo cell death or in the process of undergoing cell death, prevent, delay, or rescue the cell from death relative to a corresponding cell which has not been contacted with one of the three compounds. In particular, these compounds may act to prevent, delay, ameliorate, inhibit, reduce, or rescue neuronal cell death (e.g. apoptosis, necrosis and related cellular events).

Another aspect of the present invention is a method of preventing, inhibiting, or treating cells predisposed to undergo cell death or in the process of undergoing cell death by administering an effective amount of (RS)-(Tetrazol-5-yl)glycine, neriifolin, or cevadine to a patient in need thereof. An effective amount of (RS)-(Tetrazol-5-yl)glycine, neriifolin, or cevadine prevents, delays, ameliorates, inhibits, reduces, or rescues the cells from death relative to a corresponding cell into which this compound has not been administered.

A second aspect of the present invention is a method of treating symptoms or conditions, disorders, or diseases involving cell death by administering an effective amount of (RS)-(Tetrazol-5-yl)glycine, neriifolin, or cevadine to a patient in need thereof. An effective amount of (RS)-(Tetrazol-5-yl)glycine, neriifolin, or cevadine prevents, delays, ameliorates, inhibits, reduces, or rescues cells from death relative to a corresponding cell into which this compound has not been administered.

Another aspect of the present invention is a method of preventing, inhibiting, or treating neuronal cell death by administering an effective amount of (RS)-(Tetrazol-5-yl)glycine, neriifolin, or cevadine to a patient in need thereof. An effective amount of (RS)-(Tetrazol-5-yl)glycine, neriifolin, or cevadine prevents, delays, ameliorates, inhibits, reduces, or rescues the neuronal cells from death relative to a corresponding cell into which this compound has not been administered.

In one embodiment, (RS)-(Tetrazol-5-yl)glycine, neriifolin, and cevadine are utilized for the treatment of the types of conditions, disorders, or diseases, which can be prevented, delayed or rescued from cell death and include, but are not limited to, those associated with the central nervous system including neurological and psychiatric conditions, disorders, or diseases and those of the peripheral nervous system.

Definitions:

Unless otherwise stated, the following term used in the specification and claims have the meanings given below:

“Cell death”, as used herein, refers to any mechanism and/or pathway whereby a cell undergoes a series of events which ultimately would lead to the death of the cell. For example, cell death may be caused by various processes including, but not limited to, apoptosis or programmed cell death, necrosis, or an as yet unidentified cell death pathway. Cell death may be induced in individual cells as a consequence of numerous internal and external stimuli including, but not limited to, genetic predisposition, toxic chemicals or processes, heat, cold, rapid environmental changes, radiation, viruses, prions, bacteria, disruption of nutrient balance, or exposure to bi-products and signaling from other cells undergoing cell death. The compounds disclosed herein, when contacted with a cell (e.g. a neuronal cell) which has undergone an event that would ultimately lead to cell death (e.g. ischemia), are capable of rescuing the cell from cell death. Moreover, when a cell, into which a reporter gene (e.g. green fluorescent protein) has been introduced, undergoes an event that would likely cause cell death and subsequently the cell is contacted with a compound, expression of the reporter gene is an indication that the compound is capable of rescuing the cell from cell death.

### BRIEF DESCRIPTION OF THE DRAWINGS

FIG. 1 is a bar graph of concentration of (RS)-(Tetrazol-5-yl)glycine versus surviving neurons in a non-stroke and stroked brain slice exposed to varying concentrations of (RS)-(Tetrazol-5-yl)glycine.

FIG. 2 is a bar graph of concentration of neriifolin versus surviving neurons in a non-stroke and stroked brain slice exposed to varying concentrations of neriifolin.

FIG. 3 is a bar graph of concentration of cevadine versus surviving neurons in a non-stroke and stroked brain slice exposed to varying concentrations of cevadine.

FIG. 4 is a bar graph of concentration of sodium ascorbate versus surviving neurons in a non-stroke brain slice exposed to varying concentrations of sodium ascorbate.

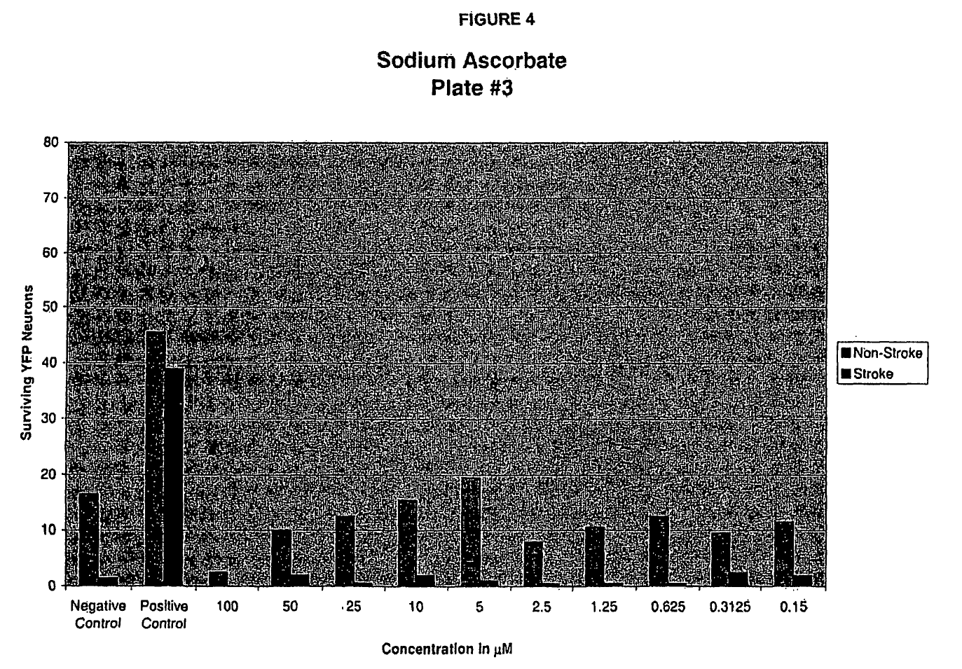

### DETAILED DESCRIPTION OF THE ILLUSTRATIVE EMBODIMENTS

The present invention relates to three compounds that exhibit protective activity against cell death, in particular neuroprotective activity. The three compounds that have been discovered to have protective activity, in particular neuroprotective activity are as follows: (RS)-(Tetrazol-5-yl)glycine, neriifolin, and cevadine. These three compounds display activity in the treatment of conditions, disorders, or diseases involving cell death, in particular neuronal cell death. These three compounds have been shown to prevent, delay, or rescue cell death in cells predisposed for undergoing cell death, whether the pathway that leads to cell death involves apoptosis, necrosis, or as yet undefined pathway.

The chemical structure of (RS)-(Tetrazol-5-yl)glycine is as follows: 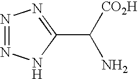
It has suprisingly been discovered that (RS)-(Tetrazol-5-yl)glycine has protective, in particular neuroprotective, activity. (RS)-(Tetrazol-5-yl)glycine may be obtained commercially or may be synthesized as described in “D,L-Tetrazol-5-ylglycine, a Highly Potent NMDA Agonist; Its Synthesis and NMDA Receptor Efficacy,” Lunn, W. H. W., et al., J. Med. Chem., 1992, 35 4608-4612, the contents of which are herein incorporated by reference in their entirety.

The chemical structure of cevadine is as follows: 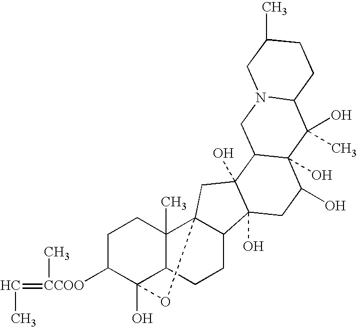
It has surprisingly been discovered that cevadine also has protective, in particular neuroprotective, activity. Cevadine is a natural product and may be isolated from the seeds of Schoenocaulon offichinale, or cevadine may be obtained commercially.

The chemical structure neriifolin is as follows: 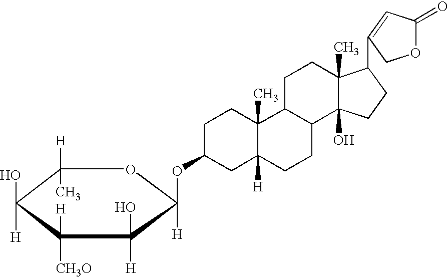
Neriifolin has surprisingly been discovered to have protective, in particular neuroprotective, activity. Neriifolin is a natural product and may be isolated from the seeds of Thevetia thevetioides, or neriifolin may be obtained commercially.

This application relates to methods for the treatment of conditions, disorders, or diseases involving cell death, in particular neuronal cells. Such applications include, the prophylactic or therapeutic use of (RS)-(Tetrazol-5-yl)glycine, cevadine, and neriifolin. When one or more of these compounds is contacted with a cell predisposed to undergo cell death or in the process of dying, these compounds prevent, delay, or rescue a cell, cells, tissue, organs, or organisms from dying. Symptoms of a condition, disorder, or disease involving cell death may be prevented, delayed, or rescued by administering one or more of (RS)-(Tetrazol-5-yl)glycine, cevadine, and neriifolin.

Accordingly, the present invention relates to methods for preventing, inhibiting, or treating cell death, in particular neuronal cell death, by administering an effective amount of one or more of the following: (RS)-(Tetrazol-5-yl)glycine, cevadine, and neriifolin.

The present invention further relates to the use of one or more of (RS)-(Tetrazol-5-yl)glycine, cevadine, and neriifolin in the production of drugs for preventing, inhibiting, or treating cell death, in particular neuronal cell death.

The present invention also relates to drugs for preventing, inhibiting, or treating cell death, in particular neuronal cell death, which contain one or more of (RS)-(Tetrazol-5-yl)glycine, cevadine, and neriifolin as the active ingredient.

Examples of Conditions, Disorders, or Diseases Involving Cell Death

The types of conditions, disorders, or diseases which can be prevented, delayed, or rescued by the compounds and methods of the present invention include, but are not limited to, those associated with the central nervous system including neurological and psychiatric conditions, disorders, or diseases; those of the peripheral nervous system; conditions, disorders, or diseases caused by physical injury; conditions, disorders, or diseases of the blood vessels or heart; conditions, disorders, or diseases of the respiratory system; neoplastic conditions, disorders, or diseases; conditions, disorders, or diseases of blood cells; conditions, disorders, or diseases of the gastrointestinal tract; conditions, disorders, or diseases of the liver; conditions, disorders, or diseases of the pancreas; conditions, disorders, or diseases of the kidney; conditions, disorders, or diseases of the ureters, urethra or bladder; conditions, disorders, or diseases of the male genital system; conditions, disorders, or diseases of the female genital tract; conditions, disorders, or diseases of the breast; conditions, disorders, or diseases of the endocrine system; conditions, disorders, or diseases of the thymus or pineal gland; conditions, disorders, or diseases of the skin or mucosa; conditions, disorders, or diseases of the musculoskeletal system; conditions, disorders, or diseases causing a fluid or hemodynamic derangement; inherited conditions, disorders, or diseases; conditions, disorders, or diseases of the immune system or spleen; conditions, disorders, or diseases caused by a nutritional disease; and conditions, disorders, or diseases typically occurring in infancy or childhood.

Conditions, disorders, or diseases involving the central nervous system include, but are not limited to, common pathophysiologic complications such as increased intracraneal pressure and cerebral herniation, septic embolism, cerebral edema, suppurative endovasculitis and hydrocephalus; infections such as meningitis, acute meningitis, acute lymphocytic meningitis, chronic meningitis, purulent meningitis, syphilitic gumma, encephalitis, cerebral abscess, epidural abscess, subdural abscess, brain abscess, viral encephalitis, acute viral encephalitis, encephalomeningitis, aseptic meningitis, post-infectious encephalitis, subacute encephalitis, chronic encephalitis, chronic meningitis, chronic encephalomeningitis, slow virus diseases and unconventional agent encephalopathies; protozoal infections such as malaria, toxoplasmosis, amebiasis and trypanosomiasis; rickettsial infections such as typhus and Rocky Mountain spotted fever; metazoal infections such as echinococcosis and cysticercosis; vascular diseases such as ischemic encephalopathy, cerebral infarction, intracranial hemorrhage, intraparenchymal hemorrhage, subarachnoid hemorrhage, mixed intraparenchymal and subarachnoid hemorrhage; conditions involving the eye such as macular degeneration, glaucoma, retinopathy of prematurity, retinitis pigmentosa, diabetic retinopathy, or other traumatic injuries to the retina or optic nerve; trauma such as epidural hematoma, subdural hematoma, parenchymal injuries; tumors such as primary intrachranial tumors, astrocytoma, oligodendroglioma, ependymoma, medulloblastoma and meningioma; degenerative diseases such as Altzheimer's disease, Huntington's disease, Parkinsonism, idiopathic Parkinson's disease and motor neuron disease; demyelinating diseases such as multiple sclerosis; nutritional, environmental and metabolic conditions, disorders, or diseases.

Conditions, disorders, or diseases of the peripheral nervous system include, but are not limited to, peripheral neuropathy, acute idiopathic polyneuropathy, diabetic neuropathy and peripheral nerve tumors.

Conditions, disorders, or diseases caused by physical injury include, but are not limited to, the direct, indirect, immediate, or delayed effects of: changes in temperature such as frostbite and thermal burns; an increase in atmospheric pressure such as air blast or immersion blast caused by an explosion; a decrease in atmospheric pressure such as caisson disease or high-altitude hypoxia; mechanical violence from penetrating or non-penetrating traumatic injury; electromechanical energy such as radiation injury from either charged particles or electromagnetic waves; electrocution or non-ionizing radiation such as radio waves, microwaves, laser light or ultrasound.

Conditions, disorders, or diseases of the blood vessels or heart include, but are not limited to, hypertension (high blood pressure), heart failure; ischemic or atherosclerotic heart disease; myocardial infarction; cardiac arrest; hypertensive heart disease; cor pulmonale; valvular heart disease such as that caused by rheumatic fever, aortic valve stenosis, mitral annulus calcification, carcinoid heart disease, nonbacterial thrombotic endocarditis, or nonbacterial verrucous endocarditis; infectious endocarditis caused by organisms including, but not limited to, Streptococcus species, Staphylococcus species, enterococci, pneumococci, gram-negative rods, Candida species, Aspergillus species, or culture-negative endocarditis; congenital heart disease such as atrial septal defect, ventricular septal defect, patent ductus arteriosis, coarctation of the aorta, Tetralogy of Fallot, tricuspid atresia, pulmonary stenosis or atresia, aortic stenosis or atresia, bicuspid aortic valve, or hypoplastic left heart syndrome; cardiomyopathy; pericarditis; pericardial effusion; rheumatoid heart disease; congenital anomalies of the blood vessels; arteriosclerosis including, but not limited to atherosclerosis, Monckeberg's medial calcific stenosis, hyaline arteriosclerosis, or hyperplastic arteriosclerosis; one or more of the vasculidities including, but not limited to, polyarteritis nodosa, hypersensitivity angiitis, Wegener's granulomatosis, giant cell (temporal) arteritis, Takayasu's arteritis, Kawasaki's disease, thromboangiitis obliterans, infectious vasculitis, Raynaud's disease; arteriosclerotic aortic aneurysm; syphilitic aortic aneurysm; dissecting aortic aneurysm; varicose veins; thrombophlebitis; lymphangitis; lymphedema; telangiectases; or arteriovenous malformations (AVM).

Conditions, disorders, or diseases of the respiratory system include, but are not limited to, pulmonary congestion; heart failure; embolism; infarction; pulmonary hypertension; adult respiratory distress syndrome (ARDS); obstructive lung disease; restrictive lung disease; chronic obstructive pulmonary disease; asthma; sarcoidosis; diffuse interstitial or infiltrative lung diseases including, but not limited to, idiopathic pulmonary fibrosis, pneumoconiosis, hypersensitivity pneumonitis, Goodpasture's syndrome, idiopathic pulmonary hemosiderosis, collagen-vascular diseases, or pulmonary eosinophilia; serofibrinous pleuritis; suppurative pleuritis; hemorrhagic pleuritis; pleural effusions; pneumothorax; hemothorax or pneumohemothorax.

Neoplastic conditions, disorders, or diseases include, but are not limited to, benign tumors composed of one parenchymal cell type such as fibromas, myxomas, lipomas, hemangiomas, meningiomas, leiomyomas, adenomas, nevi, moles, or papillomas; benign mixed tumors derived from one germ layer such as a mixed tumor of salivary gland origin; benign mixed tumors derived from more than one germ layer such as a teratoma; primary malignant tumors or metastases of malignant tumors composed of one parenchymal cell type such as sarcomas, Ewing's tumor, leukemia, myeloma, histiocytosis X, Hodgkin's disease, lymphomas, carcinomas, melanomas, bronchial adenoma, small cell lung cancer, or seminoma; primary malignant tumors or metastases of mixed malignant tumors derived from one germ layer such as Wilms' tumor or malignant mixed salivary gland tumor; primary malignant tumor or metastases of mixed malignant tumors derived from one germ layer such as malignant teratoma or teratocarcinoma; undifferentiated benign tumor or undifferentiated malignant tumor.

Conditions, disorders, or diseases of blood cells include, but are not limited to, anemia due to one or more of the following conditions: acute blood loss, chronic blood loss, hemolytic anemia, sickle cell disease, thalassemia syndromes, autoimmune hemolytic anemia, traumatic anemia, or diminished erythropoesis from megaloblastic anemia, iron deficiency, aplastic anemia, idiopathic bone marrow failure; polycythemia; hemorrhagic diatheses related to increased vascular fragility; hemorrhagic diatheses related to a reduction in platelets; idiopathic or thrombotic thrombocytopenic purpura; hemorrhagic diatheses related to defective platelet function; hemorrhagic diatheses related to abnormalities in clotting factor(s); disseminated intravascular coagulation (DIC); neutropenia; agranulocytosis; leukocytosis; plasma cell dyscrasias such as myeloma, Waldenstrom's macroglobulinemia, or heavy-chain disease; or histiocytosis.

Conditions, disorders, or diseases of the gastrointestinal tract include, but are not limited to, congenital anomalies such as atresia, fistulas, or stenosis; periodontal disease; periapical disease; xerostomia; necrotizing sialometaplasia; esophageal rings or webs; hernia; Mallory-Weiss syndrome; esophagitis; diverticulosis; diverticulitis; scleroderma; esophageal varices; acute or chronic gastritis; peptic ulcer; gastric erosion or ulceration; ischemic bowel disease; infarction; embolism; Crohn's disease; obstruction from foreign bodies, hernia, adhesion, intussusception, or volvulus; ileus; megacolon; angoidysplasia; ulcerative colitis; psuedomembranous colitis; or polyps.

Conditions, disorders, or diseases of the liver include, but are not limited to, acute hepatic failure due to one of more of metabolic, circulatory, toxic, microbial, or neoplastic causes; chronic hepatic failure due to one or more of metabolic, circulatory, toxic, microbial, or neoplastic causes; hereditary hyperbilirubinemias; infarct; embolism; hepatic circulation thrombosis or obstruction; fulminant hepatic necrosis; portal hypertension; alcoholic liver disease; post-necrotic cirrhosis; biliary cirrhosis; cirrhosis associated with alpha-1-antitrypsin deficiency; Wilson's disease; or Reye's syndrome.

Conditions, disorders, or diseases of the pancreas include, but are not limited to, congenital aberrant pancreas, congenital anomalies of pancreatic ducts, stromal fatty infiltration, pancreatic atrophy, acute hemorrhagic pancreatitis, chronic pancreatitis, chronic calcifying pancreatitis, chronic obstructive pancreatitis, pancreatic psuedocyst, diabetes mellitus, or gestational diabetes.

Conditions, disorders, or diseases of the kidney include, but are not limited to, congenital anomalies; polycystic renal disease; dialysis-associated cystic disease; glomerular disease, including, but not limited to, acute glomerulonephritis, acute proliferative glomerulonephritis, rapidly progressive glomerulonephritis, postinfectious rapidly progressive glomerulonephritis, Goodpasture's syndrome, idiopathic rapidly progressive glomerulonephritis, nephrotic syndrome, membranous glomerulonephritis, lipoid nephrosis, focal segmental glomerulosclerosis, membranoproliferative glomerulonephritis, focal proliferative glomerulonephritis, chronic glomerulonephritis, or hereditary nephritis; acute tubular necrosis; acute renal failure; tubulointerstitial diseases including, but not limited to, pyelonephritis, drug-induced interstitial nephritis, analgesic nephritis, urate nephropathy, hypercalcemia and nephrocalcinosis, hypokalemic nephropathy, myeloma-induced tubulointerstitial disease, radiation nephritis, immunologically medicated tubulointerstitial disease; hypertension; malignant hypertension; renal artery stenosis; renal diseases secondary to microangiopathic hemolytic anemia; atheroembolic renal disease; sickle cell disease nephropathy; diffuse cortical necrosis; renal infarcts; obstructive uropathy; or urolithiasis.

Conditions, disorders, or diseases of the ureters, urethra or bladder include, but are not limited to, congenital anomalies; inflammatory diseases; physical obstruction by causes including, but not limited to calculi, strictures, neoplasia, blood clot, or pregnancy; sclerosing retroperitonitis; acute cystitis; chronic cystitis; interstitial cystitis; emphysematous cystitis; eosinophilic cystitis; encrusted cystitis; fistula; or neurogenic bladder.

Conditions, disorders, or diseases of the male genital system include, but are not limited to, congenital anomalies; balanoposthitis; condyloma; phimosis; paraphimosis; dysplastic epithelial lesions; nonspecific epididymitis or orchitis; granulomatous orchitis; torsion of the testis or its vascular supply; granulomatous prostatitis; acute or chronic prostatitis; or benign prostatic hyperplasia

Conditions, disorders, or diseases of the female genital tract include, but are not limited to, congenital anomalies, lichen scleroses, acute cervicitis, chronic cervicitis, cervical polyps; acute endometritis; chronic endometritis; endometriosis; dysfunctional uterine bleeding; endometrial hyperplasia; senile cystic endometrial atrophy; salpingitis; polycystic ovary disease; pre-eclampsia or eclampsia (toxemia of pregnancy); placentitis; threatened abortion; or ectopic pregnancy.

Conditions, disorders, or diseases of the breast include, but are not limited to, congenital anomalies, acute mastitis, chronic mastitis, galactocele, granulomas, traumatic fat necrosis, mammary duct ectasia, fibrocystic disease, sclerosing adenitis, epithelial hyperplasia, hypertrophy, or gynecomastia.

Conditions, disorders, or diseases of the endocrine system include, but are not limited to, congenital anomalies; Sheehan's pituitary necrosis; empty sella syndrome; hyperthyroidism (thyrotoxicosis) from causes including, but not limited to, Graves' disease, toxic multinodular goiter, toxic adenoma, acute or subacute thyroiditis, TSH-secreting tumor, neonatal thyrotoxicosis, iatrogenic thyrotoxicosis; Hashimoto's thyroiditis; hypothyroidism (cretinism or myxedema) from causes including, but not limited to, surgical or radioactive ablation, primary idiopathic myxedema, iodine deficiency, goitrogenic agents, hypopituitarism, hypothalamic lesions, TSH resistance, subacute thyroiditis, or chronic thyroiditis; diffuse nontoxic simple or multinodular goiter; multiple endocrine neoplasia syndromes; primary or secondary hyperparathyroidism; chief cell hyperplasia; clear cell hyperplasia; hypoparathyroidism; pseudo- and pseudopseudohypoparathyrodism; Addison's disease; Waterhouse-Friderichsen syndrome; secondary adrenocortical insufficiency; Cushing's syndrome; Conn's syndrome; or congenital adrenal hyperplasia.

Conditions, disorders, or diseases of the skin or mucosa include, but are not limited to, melanocytic proliferative disorders; inflammatory dermatoses including, but not limited to, eczematous dermatitis, urticaria, erythema multiforme, cutaneous necrotizing vasculitis, cutaneous lupus erythematosus, graft-versus-host disease, panniculitis, acne vulgaris, rosacea, lichen planus, lichen sclerosus et atrophicus, pityriasis, psoriasis, or parapsoriasis; blistering diseases including, but not limited to, pemphigus, bullous pemphigoid, dermatitis herpetiformis, or porphyria.

Conditions, disorders, or diseases of the musculoskeletal system include, but are not limited to, muscular atrophy; segmental necrosis; myositis; muscular dystrophy, including, but not limited to, Duchenne type, Becker type, Fascioscapulohumeral, Limb-Girdle, myotonic dystrophy, or ocular myopathy; congenital myopathies; myasthenia gravis; traumatic myositis ossificans; nodular fasciitis; desmoid tumors; palmar fibromatosis; congenital bone disorders including, but not limited to, osteogenesis imperfecta, achondroplasia, osteopetrosis, osteochondromatosis, endochondromatosis; osteomyelitis; fractures; osteoporosis; osteomalacia; bony changes secondary to hyperparathyroidism; Paget's disease; hypertrophic osteoarthropathy; fibrous dysplasia; or nonossifying fibroma.

Conditions, disorders, or diseases causing a fluid or hemodynamic derangement include, but are not limited to, systemic edema; anasarca; edema from increased hydrostatic pressure including, but not limited to congestive heart failure, cirrhosis of the liver, constrictive pericarditis, venous obstruction; edema from reduced oncotic pressure including, but not limited to, cirrhosis of the liver, malnutrition, protein-losing renal disease, protein-losing gastroenteropathy, protein loss through increased vascular permeability; edema from lymphatic obstruction including, but not limited to, cancer, inflammatory injury, surgical injury, traumatic injury, or radiation injury; edema from increased osmotic tension in the interstitial fluid including, but not limited to, sodium retention from excessive salt intake or increased renal sodium retention, reduced renal perfusion, acute or chronic renal failure, acute or chronic renal insufficiency; edema from increased endothelial permeability including, but not limited to, inflammation, shock, burns, trauma, allergic reaction, immunologic reaction, or adult respiratory distress syndrome; ascites; pericardial effusion; hydrothorax; hyperemia; hemorrhage; mural thrombus or occlusive thrombus diminishing or obstructing vascular flow; phlebothrombosis; blood clot; embolism; thromboembolism; disseminated intravascular coagulation (DIC); amniotic fluid infusion; amniotic fluid embolism; systemic embolism disease; septic embolism; fat embolism; pulmonary embolism; air gas embolism (caisson disease or decompression sickness); anemic (white) infarction; hemorrhagic (red) infarction; cerebral infarction; septic infarction; ischemia; cardiogenic shock from conditions including, but not limited to, myocardial infarction, cardiac arrest, cardiac rupture, cardiac tamponade, pulmonary embolism, cardiac valvular obstruction, or cardiac arrhythmias; hypovolemic shock from conditions including, but not limited to, hemorrhage, vomiting, diarrhea, diaphoresis, extensive injury to bone or soft tissues, burns, or accumulation of intraperitoneal fluid; shock due to peripheral blood pooling from conditions including, but not limited to, spinal cord injury, general anesthesia, regional anesthesia, local anesthesia, drug-induced ganglionic or adrenergic blockade, gram-negative septicemia, or gram-positive septicemia; anaphylaxis, or disseminated intravascular coagulation (DIC).

Inherited conditions, disorders, or diseases include, but are not limited to, Down's syndrome, Edwards' syndrome, Patau's syndrome, other trisomies, Cri du Chat syndrome, Kinefelter's syndrome, XYY syndrome, Turner's syndrome, Multi-X female syndrome, hermaphrodism or pseudohermaphrodism, Marfan's syndrome, neurofibromatosis, vonHippel-Lindau disease, familial hypercholesterolemia, albinism, alkaptonuria, Fabry's disease, Fragile-X syndrome, Ehlers-Danlos syndromes, inherited neoplastic syndromes, inherited autosomal dominant conditions, Huntington's disease, Alport's disease, sickle-cell disease, thalessemia, tuberous sclerosis, vonWillebrand's disease, polycystic kidney disease, Pompe's disease, GM1-gangliosidosis; Tay-Sachs disease, Sandhoff-Jatzkewitz disease, metachromatic leukodystrophy, multiple sufatase deficiency, Krabbe's disease, Gaucher's disease, Niemann-Pick disease, all types of mucopolysaccharidoses, 1-cell disease, Hurler's polydystrophy, fucosidosis, mannosidosis, aspartylglycosaminuria, Wolman's disease, or acid phosphatase deficiency, inherited autosomal recessive conditions, inherited sex-linked conditions.

Conditions, disorders, or diseases of the immune system or spleen include, but are not limited to, Type I hypersensitivity conditions (anaphylaxis and other basophil or mast cell mediated conditions), Type II hypersensitivity conditions (cytotoxic conditions involving phagocytosis or lysis of target cell), Type III hypersensitivity conditions (immune complex conditions involving antigen-antibody complexes), Type IV hypersensitivity conditions (cell-mediated conditions), transplant rejection, systemic lupus erythematosus, Sjogren's syndrome, CREST, scleroderma, polymyositis-dermatomyositis, mixed connective tissue disease, polyarteritis nodosa, amyloidosis, X-linked agammaglobulinemia, comrnmon variable immunodeficiency, isolated IgA deficiency, DiGeorge's syndrome, severe combined immunodeficiency, Wiscott-Aldrich syndrome, infection with HIV virus, acquired immune deficiency syndrome (AIDS), congenital anomalies of the immune system, hypersplenism, splenomegaly, congenital anomalies of the spleen, congestive splenomegaly, infarcts, or splenic rupture.

Conditions, disorders, or diseases caused by a nutritional disease include, but are not limited to, marasmus, kwashiorkor, fat-soluble vitamin deficiency or toxicity (Vitamins A, D, E, or K), water-soluble vitamin deficiency or toxicity (thiamine, riboflavin, niacin, pyridoxine, folate, cobalamin, Vitamin C), mineral deficiency or toxicity (iron, calcium, magnesium, sodium, potassium, chloride, zinc, copper, iodine, cobalt, chromium, selenium, nickel, vanadium, manganese, molybdenum, rickets, osteomalacia, beriberi, hypoprothrombinemia, pellagra, megaloblastic anemia, scurvy, pernicious anemia, lack of gastric intrinsic factor, removal or pathophysiological functioning in the terminal ileum, microcytic anemia, or obesity.

Conditions, disorders, or diseases typically occurring in infancy or childhood include, but are not limited to, preterm birth, congenital malformations from genetic causes, congenital malformations from infectious causes, congenital malformations from toxic or teratogenic causes, congenital malformations from radiation, congenital malformations from idiopathic causes, small for gestational age infants, perinatal trauma, perinatal asphyxia, perinatal ischemia or hypoxia, birth injury, intracranial hemorrhage, deformations, respiratory distress syndrome of the newborn, atelectasis, hemolytic disease of the newborn, kernicterus, hydrops fetalis, congenital anemia of the newborn, icterus gravis, phenylketonuria, galactosemia, cystic fibrosis, hamartoma, or choristoma.

In another embodiment, the compounds and methods of the invention can be used to treat infections that cause cell death. The infections may be caused by bacteria; viruses; members of the family rickettsiae or chlamydia; fingi, yeast, hyphae or pseudohyphae; prions; protozoas; or metazoas.

Examples of aerobic or anaerobic bacteria which may cause such infections include, but are not limited to, gram-positive cocci, gram-positive bacilli (gram-positive rods), gram-negative cocci, gram-negative bacilli (gram-negative rods), Mycoplasma species, Ureaplasma species, Treponema species, Leptospira species, Borrelia species, Vibrio species, Mycobacteria species, members of Actinomycetes or L-forms (cell-wall deficient forms).

Examples of DNA, RNA or both DNA and RNA viruses which may cause such infections include, but are not limited to, members of the families adenoviridae, parvoviridae, papovaviridae, herpesviridae, poxyiridae, picornaviridae, orthomyxoviridae, paramyxoviridae, rhabdoviridae, bunyaviridae, arenaviridae, coronaviridae, retroviridae, reoviridae, togaviridae and caliciviridae.

Examples of members of the families rickettsiae or chlamydiae which may cause such infections include, but are not limited to, Rickettsia species, Rochalimaea species, Coxiella species or Chlamydia species.

Examples of fingi, yeast, hyphae or pseudohyphae which may cause such infections include, but are not limited to, members of Ascomycota, Basidiomycota, Zygomycota, or Deutoeromycota (Fungi Imperfecti); Candida species, Cryptococcus species, Torulopsis species, Rhodotorula species, Sporothrix species, Phialophora species, Cladosporium species, Xylohypha species, Blastomyces species, Histoplasma species, Coccidioides species, Paracoccidioides species, Geotrichum species, Aspergillus species, Rhizopus species, Mucor species, Pseudoallescheria species or Absidia species.

Examples of prions which may cause such infections include, but are not limited to, the causative agent of Creutzfeldt-Jakob Disease, the causative agent of Gerstmann-Straussler-Scheinker Disease, the causative agent of fatal familial insomnia, the causative agent of kuru, and the causative agent of bovine spongiform encephalopathy.

Examples of protozoa at any point in their life cycle which may cause such infections include, but are not limited to, Entamoeba species, Naegleria species, Acanthamoeba species, Pneumocystis species, Balantidium species, members of order Leptomyxida, Plasmodium species, Toxoplasma species, Leishmania species and Trypanosoma species.

Examples of metazoa at any point in their life cycle which may cause such infections include, but are not limited to, members of Platyhelminthes such as the organisms in Cestoda (tapeworrns) or Trematoda (flukes); or members of Aschelminthes such as the organisms in Acanthocephala, Chaetognatha, Cycliophora, Gastrotricha, Nematoda or Rotifera.

In a further embodiment, the compounds and methods of the invention can be used to treat infections or disorders which cause cell death in organ systems including, but not limited to, blood vessels, heart, red blood cells, white blood cells, lymph nodes, spleen, respiratory system, oral cavity, gastrointestinal tract, liver and biliary tract, pancreas, kidney, lower urinary tract, upper urinary tract and bladder, male sexual organs and genitalia, female sexual organs and genitalia, breast, thyroid gland, adrenal gland, parathyroid gland, skin, musculoskeletal system, bone marrow or bones.

In a further embodiment, the compounds and methods of the invention can be used to treat further physiological impacts on organs caused by the infections which induce cell death including, but not limited to, fever equal to or greater than 101.5 degrees Fahrenheit, a decrease or increase in pulse rate by more than 20 beats per minute, a decrease or increase in supine systolic blood pressure by more than 30 millimeters of mercury, an increase or decrease in respiratory rate by more than 8 breaths per minute, an increase or decrease in blood pH by more than 0.10 pH units, an increase or decrease in one or more serum electrolytes outside of the clinical laboratory's usual reference range, an increase or decrease in the partial pressure of arterial oxygen or carbon dioxide outside of the clinical laboratory's usual reference range, an increase or decrease in white or red blood cells outside of the laboratory's usual reference range, an acute confusional state such as delirium where delirium is defined by the American Psychiatric Association's DSM-IV Manual or a diminished level of consciousness or attention.

Pharmaceutical Preparations and Methods of Administration

The compounds, (RS)-(Tetrazol-5-yl)glycine, cevadine, and neriifolin, can be administered to a patient at therapeutically effective doses to treat or ameliorate a condition, disorder, or disease involving cell death or modulate a cell death-related process described herein. A therapeutically effective dose refers to that amount of the compound sufficient to result in amelioration of symptoms of such a condition, disorder, or disease.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds which exhibit toxic side effects may be used, care should be taken to design a delivery system which targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of the compound (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or condition, disorder, or disease, previous treatments, the general health and/or age of the subject, and other diseases present. Moreover, treatment of a subject with a therapeutically effective amount of the compounds can include a single treatment or, preferably, can include a series of treatments. In a preferred example, a subject is treated with the compound in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of the compound used for treatment may increase or decrease over the course of a particular treatment. Changes in dosage may result and become apparent from the results of diagnostic assays as described herein.

Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral rectal or topical administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXAMPLES

The invention will be further explained by the following illustrative examples which are intended to be non-limiting.

### Example 1

### Neuroprotective Effect of (RS)-(Tetrazol-5-yl)glycine

The (RS)-(Tetrazol-5-yl)glycine was obtained from Tocris Cookson, Inc., Ballwin, Mo.

Two sets of brain slices were transfected with a plasmid containing a reporter gene (e.g. a green fluorescent protein). The two sets of brain slices were exposed to a varying concentrations of (RS)-(Tetrazol-5-yl)glycine. One of the sets of brain slices was treated under conditions to simulate stroke, and the other brain slice was a non-stroke control. Survival of neurons in the brain slices, as indicated by expression of the reporter molecule, was examined by direct neuron counts after 24 hrs. exposure to the compound. Since the (RS)-(Tetrazol-5-yl)glycine exhibits neuroprotective properties, transfected neurons may be counted in both the stroke and non-stroke brain slices.

A negative and positive control experiments were also run for comparison. In the positive control, a known neuroprotective gene and reporter gene (i.e., a green fluorescent protein) were shot into the stroke and non-stroke brain slices. In the negative control, only the reporter gene (i.e., a green fluorescent protein) was shot into the stroke and non-stroke brain slices.

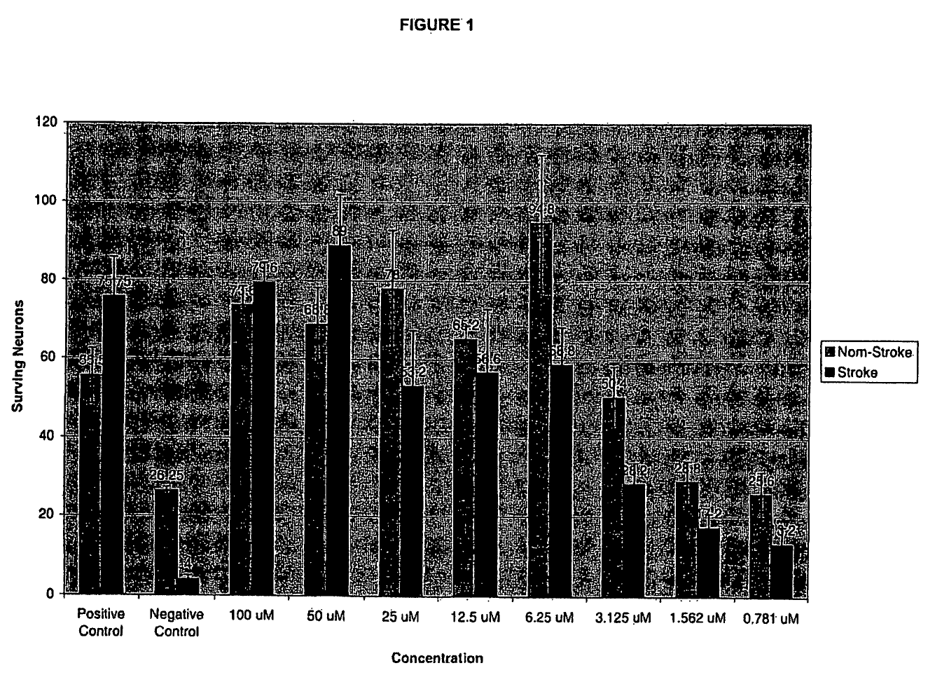

The results of the runs using varying concentrations of (RS)-(Tetrazol-5-yl)glycine are summarized numerically below in Table I and graphically in FIG. 1.

TABLE I
POSITIVE CONTROL

| | NON-STROKE | STROKE |
|---|---|---|
| neurons | 46 | 68 |
| neurons | 42 | 98 |
| neurons | 62 | 51 |
| neurons | 72 | 86 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 55.5 | 75.75 | 13.9881 | 20.5973 | 6.99405 | 10.2987 |

NEGATIVE CONTROL

| | NON-STROKE | STROKE |
|---|---|---|
| neurons | 26 | 4 |
| neurons | 29 | 2 |
| neurons | 24 | 3 |
| neurons | 26 | 7 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 26.25 | 4 | 2.06155 | 2.16025 | 1.03078 | 1.08012 |

100 uM (RS)-(Tetrazol-5-yl)glycine

| | NON-STROKE | STROKE |
|---|---|---|
| neurons | 72 | 95 |
| neurons | 87 | 76 |
| neurons | 64 | 87 |
| neurons | 62 | 66 |
| neurons | 83 | 74 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 73.6 | 79.6 | 11.149 | 11.4149 | 4.98598 | 5.1049 |

50 uM (RS)-(Tetrazol-5-yl)glycine

| | NON-STROKE | STROKE |
|---|---|---|
| neurons | 58 | 101 |
| neurons | 45 | 94 |
| neurons | 96 | 126 |
| neurons | 59 | 76 |
| neurons | 86 | 48 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 68.8 | 89 | 21.3002 | 29.1033 | 9.52575 | 13.0154 |

25 uM (RS)-(Tetrazol-5-yl)glycine

| | NON-STROKE | STROKE |
|---|---|---|
| neurons | 83 | 47 |
| neurons | 46 | 95 |
| neurons | 72 | 74 |
| neurons | 132 | 36 |
| neurons | 57 | 14 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 78 | 53.2 | 33.3242 | 31.8229 | 14.903 | 14.2317 |

12.5 uM (RS)-(Tetrazol-5-yl)glycine

| | NON-STROKE | STROKE |
|---|---|---|
| neurons | 71 | 42 |
| neurons | 61 | 33 |
| neurons | 75 | 42 |
| neurons | 56 | 121 |
| neurons | 63 | 45 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 65.2 | 56.6 | 7.69415 | 36.2808 | 3.44093 | 16.2253 |

6.25 uM (RS)-(Tetrazol-5-yl)glycine

| | NON-STROKE | STROKE |
|---|---|---|
| neurons | 58 | 58 |
| neurons | 126 | 25 |
| neurons | 146 | 78 |
| neurons | 62 | 76 |
| neurons | 82 | 57 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 94.8 | 58.8 | 39.3345 | 21.2767 | 17.5909 | 9.51525 |

3.125 uM (RS)-(Tetrazol-5-yl)glycine

| | NON-STROKE | STROKE |
|---|---|---|
| neurons | 56 | 45 |
| neurons | 26 | 16 |
| neurons | 46 | 16 |
| neurons | 75 | 31 |
| neurons | 49 | 33 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 50.4 | 28.2 | 17.7003 | 12.3572 | 7.91581 | 5.5263 |

1.562 uM (RS)-(Tetrazol-5-yl)glycine

| | NON-STROKE | STROKE |
|---|---|---|
| neurons | 36 | 23 |
| neurons | 19 | 14 |
| neurons | 26 | 10 |
| neurons | 46 | 28 |
| neurons | 17 | 11 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 28.8 | 17.2 | 12.1532 | 7.91833 | 5.43507 | 3.54119 |

0.781 uM (RS)-(Tetrazol-5-yl)glycine

| | NON-STROKE | STROKE |
|---|---|---|
| neurons | 35 | 10 |
| neurons | 23 | 2 |
| neurons | 5 | 28 |
| neurons | 37 | 14 |
| neurons | 28 | 12 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 25.6 | 13.2 | 12.7984 | 9.44458 | 5.72364 | 4.22374 |

### Example 2

### Neuroprotective Effect of Neriifolin

Neriifolin was obtained from Microsource Discovery Systems, Inc., Gaylordsville, Conn.

Two sets of brain slices were transfected with a plasmid containing a reporter gene (e.g. a green fluorescent protein). The two set of brain slices were exposed to a varying concentrations of neriifolin. One of the sets of brain slices was treated under conditions to simulate stroke, and the other brain slice was a non-stroke control. Survival of neurons in the brain slices, as indicated by expression of the reporter molecule, was examined by direct neuron counts after 24 hrs. exposure to the compound. Since the neriifolin exhibits neuroprotective properties, transfected neurons may be counted in both the stroke and non-stroke brain slices.

A negative and positive control experiments were also run for comparison as described above for Example 1.

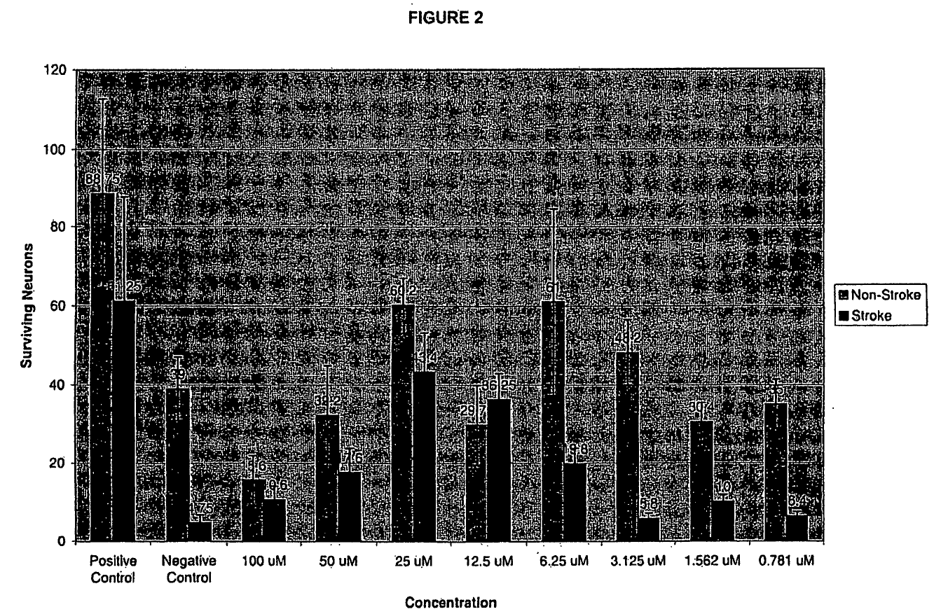

The results of the runs using varying concentrations of neriifolin are summarized numerically below in Table II and graphically in FIG. 2.

TABLE II
POSITIVE CONTROL

| | NON-STROKE | STROKE |
|---|---|---|
| neurons | 93 | 60 |
| neurons | 37 | 7 |
| neurons | 73 | 44 |
| neurons | 152 | 134 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 88.75 | 61.25 | 48.1136 | 53.3378 | 24.0568 | 26.6689 |

NEGATIVE CONTROL

| | NON-STROKE | STROKE |
|---|---|---|
| neurons | 17 | 0 |
| neurons | 44 | 4 |
| neurons | 56 | 5 |
| neurons | 39 | 10 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 39 | 4.75 | 16.3095 | 4.11299 | 8.15475 | 2.05649 |

100 uM Neriifolin

| NON-STROKE | STROKE |
|---|---|
| 9 | 2 |
| 11 | 7 |
| 8 | 8 |
| 9 | 22 |
| 41 | 14 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 15.6 | 10.6 | 14.2408 | 7.66812 | 6.36867 | 3.42929 |

50 uM Neriifolin

| | NON-STROKE | STROKE |
|---|---|---|
| neurons | 17 | 12 |
| neurons | 3 | 5 |
| neurons | 64 | 19 |
| neurons | 62 | 41 |
| neurons | 15 | 11 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 32.2 | 17.6 | 28.6304 | 13.9929 | 12.8039 | 6.2578 |

25 uM Neriifolin

| | NON-STROKE | STROKE |
|---|---|---|
| neurons | 49 | 30 |
| neurons | 66 | 46 |
| neurons | 82 | 13 |
| neurons | 42 | 68 |
| neurons | 62 | 60 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 60.2 | 43.4 | 15.5628 | 22.3114 | 6.95989 | 9.97798 |

12.5 uM Neriifolin

| | NON-STROKE | STROKE |
|---|---|---|
| neurons | 53 | 58 |
| neurons | 0 | 24 |
| neurons | 25 | 32 |
| neurons | 41 | 31 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 29.75 | 36.25 | 22.9111 | 14.9304 | 10.2461 | 6.67708 |

6.25 uM Neriifolin

| | NON-STROKE | STROKE |
|---|---|---|
| neurons | 154 | 26 |
| neurons | 34 | 19 |
| neurons | 28 | 13 |
| neurons | 56 | 31 |
| neurons | 33 | 10 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 61 | 19.8 | 53.0943 | 8.75785 | 23.7445 | 3.91663 |

3.125 uM Neriifolin

| | NON-STROKE | STROKE |
|---|---|---|
| neurons | 78 | 4 |
| neurons | 53 | 9 |
| neurons | 31 | 2 |
| neurons | 36 | 9 |
| neurons | 43 | 5 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 48.2 | 5.8 | 18.593 | 3.11448 | 8.31505 | 1.39284 |

1.562 uM Neriifolin

| | NON-STROKE | STROKE |
|---|---|---|
| neurons | 48 | 9 |
| neurons | 34 | 10 |
| neurons | 26 | 13 |
| neurons | 19 | 14 |
| neurons | 25 | 4 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 30.4 | 10 | 11.1937 | 3.937 | 5.006 | 1.76068 |

0.781 uM Neriifolin

| | NON-STROKE | STROKE |
|---|---|---|
| neurons | 29 | 10 |
| neurons | 38 | 6 |
| neurons | 16 | 8 |
| neurons | 55 | 4 |
| neurons | 37 | 4 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 35 | 6.4 | 14.2302 | 2.60768 | 6.36396 | 1.16619 |

### Example 3

### Neuroprotective Effect of Cevadine

Cevadine was obtained from Microsource Discovery Systems, Inc., Gaylordsville, Conn.

Two sets of brain slices were transfected with a plasmid containing a reporter gene (e.g. a green fluorescent protein). The two sets of brain slices were exposed to a varying concentrations of cevadine. One of the sets of brain slices was treated under conditions to simulate stroke, and the other brain slice was a non-stroke control. Survival of neurons in the brain slices, as indicated by expression of the reporter molecule, was examined by direct neuron counts after 24 hrs. exposure to the compound. Since the cevadine exhibits neuroprotective properties, transfected neurons may be counted in both the stroke and non-stroke brain slices.

A negative and positive control experiments were also run for comparison as described above for Example 1.

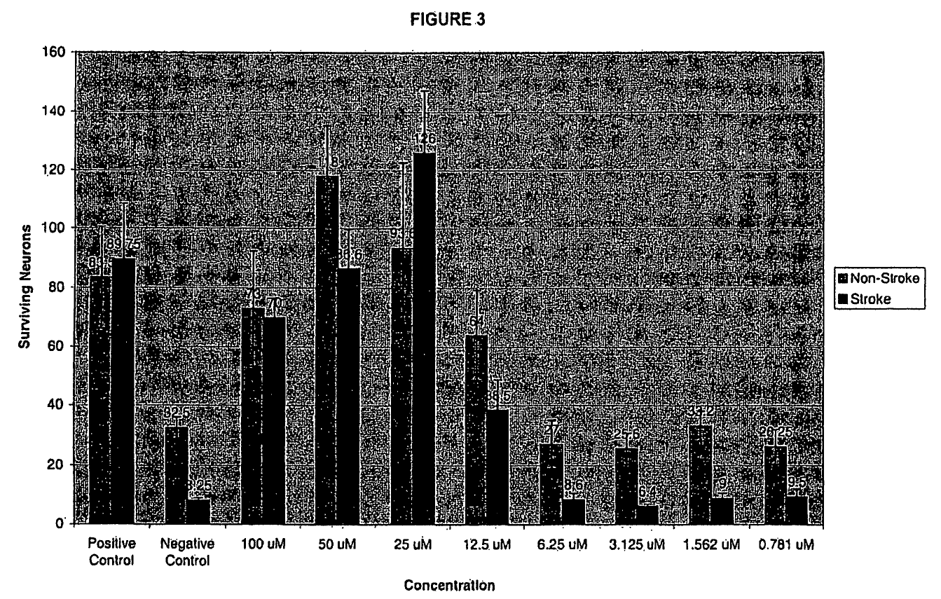

The results of the runs using varying concentrations of cevadine are summarized numerically below in Table III and graphically in FIG. 3.

POSITIVE CONTROL

| | NON-STROKE | STROKE |
|---|---|---|
| neurons | 110 | 131 |
| neurons | 115 | 113 |
| neurons | 46 | 61 |
| neurons | 63 | 54 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 83.5 | 89.75 | 34.2588 | 38.0646 | No Data | No Data |

NEGATIVE CONTROL

| | NON-STROKE | STROKE |
|---|---|---|
| neurons | 39 | 9 |
| neurons | 35 | 10 |
| neurons | 22 | 8 |
| neurons | 34 | 6 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 32.5 | 8.25 | 7.32575 | 1.70783 | No Data | No Data |

100 uM Cevadine

| | NON-STROKE | STROKE |
|---|---|---|
| neurons | 37 | 53 |
| neurons | 85 | 78 |
| neurons | 53 | 77 |
| neurons | 46 | 56 |
| neurons | 144 | 86 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 73 | 70 | 43.6177 | 14.6116 | No Data | No Data |

50 uM Cevadine

| | NON-STROKE | STROKE |
|---|---|---|
| neurons | 128 | 132 |
| neurons | 65 | 68 |
| neurons | 166 | 57 |
| neurons | 135 | 98 |
| neurons | 96 | 78 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 118 | 86.6 | 38.6846 | 29.5263 | No Data | No Data |

25 uM Cevadine

| | NON-STROKE | STROKE |
|---|---|---|
| neurons | 86 | 149 |
| neurons | 43 | 53 |
| neurons | 114 | 118 |
| neurons | 31 | 182 |
| neurons | 194 | 128 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 93.6 | 126 | 65.2557 | 47.5973 | No Data | No Data |

12.5 uM Cevadine

| | NON-STROKE | STROKE |
|---|---|---|
| neurons | 82 | 24 |
| neurons | 14 | 16 |
| neurons | 71 | 46 |
| neurons | 89 | 68 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 64 | 38.5 | 34.1467 | 23.4023 | No Data | No Data |

6.25 uM Cevadine

| | NON-STROKE | STROKE |
|---|---|---|
| neurons | 23 | 4 |
| neurons | 25 | 15 |
| neurons | 20 | 12 |
| neurons | 58 | 8 |
| neurons | 9 | 4 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 27 | 8.6 | 18.3984 | 4.87852 | No Data | No Data |

3.125 uM Cevadine

| | NON-STROKE | STROKE |
|---|---|---|
| neurons | 28 | 9 |
| neurons | 33 | 5 |
| neurons | 26 | 6 |
| neurons | 6 | 7 |
| neurons | 35 | 5 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 25.6 | 6.4 | 11.5456 | 1.67332 | No Data | No Data |

1.562 uM Cevadine

| | NON-STROKE | STROKE |
|---|---|---|
| neurons | 16 | 7 |
| neurons | 45 | 8 |
| neurons | 30 | 7 |
| neurons | 36 | 16 |
| neurons | 39 | 7 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 33.2 | 9 | 11.0318 | 3.937 | No Data | No Data |

0.781 uM Cevadine

| | NON-STROKE | STROKE |
|---|---|---|
| neurons | 37 | 14 |
| neurons | 38 | 2 |
| neurons | 14 | 8 |
| neurons | 16 | 14 |

| | | NON- | | NON- | |
|---|---|---|---|---|---|
| NON- | | STROKE | STROKE | STROKE | STROKE |
| STROKE | STROKE | STD. | STD. | STD. | STD. |
| AVERAGE | AVG. | DEV. | DEV. | ERROR | ERROR |
| 26.25 | 9.5 | 13.0224 | 5.74456 | No Data | No Data |

### Comparative Example

Sodium ascorbate was obtained from Sigma Chemicals of St. Louis, Mo.

Two sets of brain slices were transfected with a plasmid containing a reporter gene (e.g. a green fluorescent protein). The two sets of brain slices were exposed to a varying concentrations of sodium ascorbate. One of the sets of brain slices was treated under conditions to simulate stroke, and the other brain slice was a non-stroke control. Survival of neurons in the brain slices, as indicated by expression of the reporter molecule, was examined by direct neuron counts after 24 hrs. exposure to the compound. Since the sodium ascorbate did not exhibit neuroprotective properties, few transfected neurons may be counted in the stroke brain slices.

A negative and positive control experiments were also run for comparison as described above for Example 1.

The results of the runs using varying concentrations of sodium ascorbate are summarized numerically below in Table IV.

TABLE IV
POSITIVE CONTROL

| | NON-STROKE | STROKE |
|---|---|---|
| neurons | 51 | 49 |
| neurons | 40 | 29 |

| NON-STROKE AVERAGE | STROKE AVG. |
|---|---|
| 45.5 | 39 |

NEGATIVE CONTROL

| | NON-STROKE | STROKE |
|---|---|---|
| neurons | 20 | 1 |
| neurons | 13 | 2 |

| NON-STROKE AVERAGE | STROKE AVG. |
|---|---|
| 16.5 | 1.5 |

100 uM Sodium Ascorbate

| | NON-STROKE | STROKE |
|---|---|---|
| neurons | 4 | 0 |
| neurons | 1 | 0 |

| NON-STROKE AVERAGE | STROKE AVG. |
|---|---|
| 2.5 | 0 |

50 uM Sodium Ascorbate

| | NON-STROKE | STROKE |
|---|---|---|
| neurons | 13 | 1 |
| neurons | 7 | 3 |

| NON-STROKE AVERAGE | STROKE AVG. |
|---|---|
| 10 | 2 |

25 uM Sodium Ascorbate

| | NON-STROKE | STROKE |
|---|---|---|
| neurons | 12 | 0 |
| neurons | 13 | 1 |

| NON-STROKE AVERAGE | STROKE AVG. |
|---|---|
| 12.5 | 0.5 |

10 uM Sodium Ascorbate

| | NON-STROKE | STROKE |
|---|---|---|
| neurons | 16 | 1 |
| neurons | 15 | 3 |

| NON-STROKE AVERAGE | STROKE AVG. |
|---|---|
| 15.5 | 2 |

5 uM Sodium Ascorbate

| | NON-STROKE | STROKE |
|---|---|---|
| neurons | 22 | 0 |
| neurons | 17 | 2 |

| NON-STROKE AVERAGE | STROKE AVG. |
|---|---|
| 19.5 | 1 |

2.5 uM Sodium Ascorbate

| | NON-STROKE | STROKE |
|---|---|---|
| neurons | 7 | 1 |
| neurons | 9 | 0 |

| NON-STROKE AVERAGE | STROKE AVG. |
|---|---|
| 8 | 0.5 |

1.25 uM Sodium Ascorbate

| | NON-STROKE | STROKE |
|---|---|---|
| neurons | 11 | 0 |
| neurons | 10 | 1 |

| NON-STROKE AVERAGE | STROKE AVG. |
|---|---|
| 10.5 | 0.5 |

0.625 uM Sodium Ascorbate

| | NON-STROKE | STROKE |
|---|---|---|
| neurons | 10 | 1 |
| neurons | 15 | 0 |

| NON-STROKE AVERAGE | STROKE AVG. |
|---|---|
| 12.5 | 0.5 |

0.3125 uM Sodium Ascorbate

| | NON-STROKE | STROKE |
|---|---|---|
| neurons | 13 | 3 |
| neurons | 6 | 2 |

| NON-STROKE AVERAGE | STROKE AVG. |
|---|---|
| 9.5 | 2.5 |

0.15 uM Sodium Ascorbate

| | NON-STROKE | STROKE |
|---|---|---|
| neurons | 12 | 4 |
| neurons | 11 | 0 |

| NON-STROKE AVERAGE | STROKE AVG. |
|---|---|
| 11.5 | 2 |

Various modifications and alterations of this invention will become apparent to those skilled in the art without departing from the scope and spirit of this invention.

1. A method of preventing, inhibiting, or treating cells predisposed to undergo cell death or in the process of undergoing cell death, the method comprising contacting the cells with an effective amount of (RS)-(Tetrazol-5-yl)glycine, neriifolin, or cevadine.

2. The method of claim 1 wherein the cells are neuronal cells.

3. A method of treating a condition, disorder, or disease involving cell death, the method comprising administering an effective amount of (RS)-(Tetrazol-5-yl)glycine, neriifolin, or cevadine to a patient in need thereof.

4. The method of claim 3 wherein the condition, disorder, or disease is associated with the central nervous system.

5. A method of preventing, inhibiting, or treating cell death, the method comprising administering an effective amount of (RS)-(Tetrazol-5-yl)glycine, neriifolin, or cevadine to a patient in need thereof.

6. The method of claim 5 wherein the cell death that is prevented, inhibited, or treated is neuronal cell death.
